(19) United States
(12) Patent Application Publication
(10) Pub. No.: US 2015/0173705 A1
(43) Pub. Date: Jun. 25, 2015

(54) APPARATUS AND METHOD FOR ADAPTING DIAGNOSTIC MODEL FOR COMPUTER-AIDED DIAGNOSIS

(71) Applicant: Samsung Electronics Co., Ltd., Suwon-si (KR)

(72) Inventors: Ki-Yong LEE, Suwon-si (KR); Yeong-Kyeong SEONG, Yongin-si (KR)

(73) Assignee: Samsung Electronics Co., Ltd., Suwon-si (KR)

(21) Appl. No.: 14/296,500

(22) Filed: Jun. 5, 2014

(30) Foreign Application Priority Data
Dec. 23, 2013 (KR) 10-2013-0161568

(51) Int. Cl. A61B 8/08; A61B 5/00; A61B 8/14; A61B 5/055; A61B 6/03

(52) U.S. Cl. CPC A61B 8/0825; A61B 5/055; A61B 6/032; A61B 8/14; A61B 5/7267

(57) ABSTRACT

An apparatus and method for adapting a diagnostic model for Computer-Aided Diagnosis (CAD). The CAD apparatus includes an image acquirer configured to acquire an image of an object, a diagnosis model store configured to store at least one diagnostic model, an adaptive information provider configured to provide adaptive information regarding the image, a diagnostic model adapter configured to select a diagnostic model from the diagnostic model store and to generate an adapted diagnostic model based on the selected diagnostic model and the adaptive information, and a diagnoser configured to perform CAD on the image using the adapted diagnostic model.

### RELATED APPLICATION

This application claims the benefit under 35 U.S.C. §119(a) of Korean Patent Application No. 10-2013-0161568, filed on Dec. 23, 2013, in the Korean Intellectual Property Office, the entire disclosure of which is incorporated herein by references for all purposes.

### BACKGROUND

1. Field

The following description relates to a model for Computer-Aided Diagnosis (CAD), and to a diagnostic model for CAD that utilizes additional information that may affect the result of the diagnosis.

2. Description of Related Art

Computer-Aided Diagnosis (CAD) is a technique of performing diagnosis on medical image data based on results of computer analysis. CAD quantitatively analyzes medical image data using pattern recognition or geometrical image processing. Such analysis may be performed by a diagnostic model that includes various parameters.

For example, a single diagnostic model is usually used for the entire area of a breast when CAD is used to analyze ultrasound images of breasts to diagnosis breast cancer. Statistically, an incidence rate of breast cancer differs among people according to where a tumor is located in a breast. In addition, gender, age, and family medical history may also affect the incidence rate. Thus, in order to improve the accuracy of diagnosis, efforts have been made to utilize such statistical or empirical information.

Conventionally, a location of a probe of an ultrasound image capturing device is included in a captured ultrasound image. Based on the location of the probe, a doctor may perform diagnosis with higher accuracy when analyzing the ultrasound image. An ultrasound image capturing device may adjust parameters of an ultrasound image based on capture information, such as a location and an angle of a probe. In this case, the ultrasound image capturing device adjusts physical parameters, such as Time Gain Compensation (TGC) and Dynamic Range (DR), according to capture information.

### SUMMARY

This Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter.

In one general aspect, there is provided a Computer-Aided Diagnosis (CAD) apparatus including an image acquirer configured to acquire an image of an object, a diagnosis model store configured to store at least one diagnostic model, an adaptive information provider configured to provide adaptive information regarding the image, a diagnostic model adapter configured to select a diagnostic model from the diagnostic model store and to generate an adapted diagnostic model based on the selected diagnostic model and the adaptive information, and a diagnoser configured to perform CAD on the image using the adapted diagnostic model.

The adaptive information may include information indicating a position of the object.

The adaptive information may include personal information of the object that affects the CAD of the images.

The adaptive information may include information indicating the performance of a specific diagnostic model corresponding to the image captured at a specific capture position.

The diagnostic model adapter may be further configured to adjust at least one parameter of the at least one diagnostic model based on the adaptive information and to provide the adjusted diagnostic model to the diagnoser.

The diagnostic model store may be further configured to store a plurality of diagnostic models that correspond to a plurality of image sets based on the adaptive information, and the diagnostic model adapter may be further configured to select one of the plurality of diagnostic models based on the adaptive information and to provide the selected diagnostic model to the diagnoser.

The diagnostic model adapter may be further configured to adjust at least one parameter of the selected diagnostic model based on the adaptive information and to provide the adjusted diagnostic model to the diagnoser.

The plurality of diagnostic models may be divided into two or more sub sets of diagnostic models based on the adaptive information, and the diagnostic models in each sub set are classified hierarchically based on the adaptive information, and wherein the diagnostic models with greater amount of adaptive information may be classified in a higher hierarchical level.

The diagnostic model adapter may be further configured to: select a highest hierarchical level diagnostic model among the plurality of diagnostic models based on a part of the adaptive information, determine performance of the selected diagnostic model based on another part of the adaptive information, in response to the performance of the selected diagnostic model not being suitable, select a lower hierarchical level diagnostic model and determine suitability of the lower hierarchical level diagnostic model, and in response to the performance of the selected diagnostic model being suitable, provide the selected diagnostic model to the diagnoser.

The apparatus may include a diagnostic model leaner configured to: classify images of an image set into sub image sets according to a learning criterion, learn a diagnostic model stored in the diagnostic model store using the classified sub image sets, and store the learned diagnostic model in the diagnostic model store.

In another general aspect, there is provided a method for adapting a diagnostic model for Computer-Aided Diagnosis (CAD), the method including: acquiring an image of an object, providing adaptive information regarding the images, acquiring a diagnostic model for diagnosis of the image, adapting, at a diagnostic model adapter, the diagnostic model based on the adaptive information, and performing CAD on the image using the adapted diagnostic model.

The adaptive information may include information indicating a position of the object.

The adaptive information may include personal information of the object that affects the CAD of the images.

The adaptive information may include information indicating the performance of a specific diagnostic model for the image captured at a specific capture position.

The adapting of the diagnostic model may include adjusting at least one parameter of the diagnostic model based on the adaptive information and providing the adjusted diagnostic model.

The adapting of the diagnostic model may include selecting one of a plurality of diagnostic models that correspond to a plurality of image sets based on the adaptive information and providing the selected diagnostic model.

The adapting of the diagnostic model may include adjusting at least one parameter of the selected diagnostic model and providing the adjusted diagnostic model.

The plurality of diagnostic models may be classified into two or more sub sets of diagnostics models based on the adaptive information, and the diagnostic models in each sub set are hierarchically classified according to the adaptive information, and the diagnostic models with greater amount of the adaptive information may be classified in a higher the hierarchical level.

The adapting of the diagnostic model may include selecting a highest hierarchical level diagnostic model from among the plurality of diagnostic models based on a part of the adaptive information, determining performance of the selected diagnostic model based on a another part of the adaptive information, in response to the performance of the selected diagnostic model not being suitable, selecting another diagnostic model with a lower hierarchical level than the hierarchical level of the selected diagnostic model and determining the suitability of the lower hierarchical level diagnostic mode, and in response to the performance of the selected diagnostic model being suitable, providing the selected diagnostic model.

The method may include classifying an image set according to a learning criterion, learning a diagnostic model stored in the diagnostic model store using the classified image set, and storing the learned diagnostic model to use the learned diagnostic model in the adapting of the diagnostic model.

In another general aspect, there is provided a diagnostic model provider for a Computer-Aided Diagnosis (CAD) system including an adaptive information provider configured to provide adaptive information regarding images of an object, a diagnostic model store configured to store a plurality of diagnostic models corresponding to the images, and a diagnostic model adapter configured to select one of the plurality of diagnostic models based on the adaptive information and to provide the selected diagnostic model for the CAD, wherein the plurality of diagnostic models are classified hierarchically based on the adaptive information and the diagnostic models with greater number of adaptive information are classified in a higher hierarchical level.

The diagnostic model adapter may be further configured to: select a highest hierarchical level diagnostic model among the plurality of diagnostic models based on a part of the adaptive information, determine performance of the selected diagnostic model based on another part of the adaptive information, select a lower hierarchical level diagnostic model, in response to the performance of the selected diagnostic model being lower than a threshold value, and provide the selected diagnostic model for the CAD, in response to the performance of the selected diagnostic model being higher than a threshold value.

Other features and aspects may be apparent from the following detailed description, the drawings, and the claims.

### BRIEF DESCRIPTION OF THE DRAWINGS

FIG. 1A is a diagram illustrating an example of a configuration of a system for adapting a diagnostic model for Computer-Aided Diagnosis (CAD).

FIG. 1B is a diagram illustrating an example of a configuration of the system 100 shown in FIG. 1A where a diagnostic model learner is included.

FIG. 2 is a diagram illustrating examples of classified capture positions included in capture information of images within a system for adapting a diagnostic model for CAD.

FIG. 3 is a diagram illustrating examples of a plurality of diagnostic models that are classified from a data set based on capture information within a system for adapting a diagnostic model for Computer-Aided Diagnosis (CAD).

FIG. 4 is a diagram illustrating examples of a plurality of diagnostic models that are hierarchically classified with respect to an image set based on capture information within a system for adapting a diagnostic model for CAD.

FIG. 5 is a diagram illustrating an example of a hierarchical structure of diagnostic models stored in the system described in FIG. 4.

FIG. 6 is a diagram illustrating an example of performing diagnosis using a general diagnostic model for Computer-Aided Diagnosis (CAD).

FIG. 7 is a diagram illustrating an example of performing diagnosis using a diagnostic model selected from different diagnostic models according to capture positions in a system for adapting a diagnostic model for CAD.

FIG. 8 is a diagram illustrating an example of performing diagnosis by adjusting parameters of a diagnostic model based on the capture position within a system for adapting a diagnostic model for CAD.

FIG. 9 is a diagram illustrating an example of performing diagnosis by determining whether to use a diagnostic model, based on a statistical result that shows diagnosis performance according to capture positions, within a system for adapting a diagnostic model for Computer-Aided Diagnosis (CAD).

FIG. 10 is a diagram illustrating an example of a process of selecting a diagnostic model based on capture information of a method for adapting a diagnostic model for CAD.

FIG. 11 is a diagram illustrating an example of a process of adjusting parameters of a diagnostic model based on capture information within a method for adapting a diagnostic model for CAD.

FIG. 12 is a diagram illustrating an example of a process of performing diagnosis by selecting a diagnostic model with decent diagnosis performance based on diagnosis performance of the diagnostic model within a method for adapting a diagnostic model for CAD.

FIG. 13 is a diagram illustrating an example of a process of performing diagnosis by selecting a diagnostic model according to a determined selection criterion within a method for adapting a diagnostic model for CAD.

FIG. 14 is a diagram illustrating an example of a process of performing diagnosis by adjusting parameters of a diagnostic model according to a determined adjustment criterion within a method for adapting a diagnostic model for CAD.

FIG. 15 is a diagram illustrating an example of a process of learning a diagnostic model within a method for adapting a diagnostic model for CAD.

FIG. 16 is a diagram illustrating an example of a process of learning a hierarchically structured diagnostic model within a method for adapting a diagnostic model for CAD.

Throughout the drawings and the detailed description, unless otherwise described, the same drawing reference numerals will be understood to refer to the same elements, features, and structures. The drawings may not be to scale, and the relative size, proportions, and depiction of elements in the drawings may be exaggerated for clarity, illustration, and convenience.

### DETAILED DESCRIPTION

The following detailed description is provided to assist the reader in gaining a comprehensive understanding of the methods, apparatuses, and/or systems described herein. However, various changes, modifications, and equivalents of the systems, apparatuses and/or methods described herein will be apparent to one of ordinary skill in the art. The progression of processing steps and/or operations described is an example; however, the sequence of and/or operations is not limited to that set forth herein and may be changed as is known in the art, with the exception of steps and/or operations necessarily occurring in a certain order. Also, descriptions of functions and constructions that are well known to one of ordinary skill in the art may be omitted for increased clarity and conciseness.

The features described herein may be embodied in different forms, and are not to be construed as being limited to the examples described herein. Rather, the examples described herein have been provided so that this disclosure will be thorough and complete, and will convey the full scope of the disclosure to one of ordinary skill in the art.

Hereinafter, a system and method for adapting a diagnostic model for Computer-Aided Diagnosis (CAD) are described with examples of images of a part of a patient's breast. It is understood that a range and a breadth of the disclosure is not limited to any portion of the body, and the systems and methods disclosed herein may be applicable to any and all portions of the body. For any object that requires medical diagnosis, it is possible to capture medical images. Thus, the system and method may be widely applied to general medical images.

In addition, the following examples are described with reference to ultrasound images, but those skilled in the art may apply the systems and methods disclosed herein to other types of images. For example, the systems and methods may also be applied to X-ray images, Computed Tomography (CT) images, and Magnetic Resonance (MR) images.

In a non-exhaustive example, the system and method may be used for CAD that is designed to analyze ultrasound medical image data, such as, ultrasound images of a part of a patient's breast to perform diagnosis for breast cancer.

In this case, the image data may be digital image that are captured by an ultrasound imaging device and then provided through systems, such as, for example, Picture Archiving and Communication System (PACS), Medical Imaging System (MIS).

To perform CAD on image data, a diagnostic model is required. The diagnostic model may refer to a group of parameters. Parameters of a diagnostic model may include image data analytic parameters that are used in pattern recognition to detect, segment and classify a Region of Interest (ROI) through analysis of image data, and in image analysis to analyze and process image data.

Parameters of a diagnostic model may be defined differently according to the purpose of a diagnosis. For example, a diagnostic model used for CAD may be defined to analyze ultrasound images of a part of breast for diagnosis of breast cancer. A conventional diagnostic model is not modified once it is applied, and thus, the diagnostic model is applied to the whole specific object, for example, breasts. However, the height of a breast is different around a nipple, and the distribution of organs inside a breast is different according to the capture positions. Considering the above characteristics, diagnostic models including different parameters may be applied for CAD according to the capture positions.

In addition, the anatomical structure of a specific object may be different for different capture positions and for different users. The anatomical structure of an object may be different even for the same person at different points in time. It is impractical to generate diagnostic models accounting for all possible differences. Even if such a detailed model is generated, the diagnostic models may be almost useless because it has a narrow range of applicability.

For these reasons, in order to enhance diagnosis accuracy, a diagnostic model should be sophisticated enough to be applied in a narrow range of applicability and should include parameters that are general enough to be applied in practice.

In the non-exhaustive examples described herein, a diagnostic model that is predetermined to be applied in a specific range is stored. When CAD is performed on specific image data, the stored diagnostic model is adapted to perform diagnosis on the specific image data with higher accuracy. Then, the adapted diagnostic model is used to perform diagnosis of the specific image data.

The diagnostic model may be adapted using capture information, information on a captured subject, and/or information on the diagnostic model itself. Accordingly, a different adapted diagnostic model may be applied to each different sub-divided area of a subject to be captured.

For example, capture position information indicating which part of a patient's body has been captured may be used as adaptive information to adapt a diagnostic model.

Adaption of diagnostic model may be performed by selecting a suitable diagnostic model based on adaptive information or by adjusting parameters of a diagnostic model. If CAD is performed on image data using the diagnostic model adapted in the above manner, diagnosis accuracy may be enhanced.

Adaptive information for a diagnostic model may include capture information. The capture information may include, for example, information that may affect a diagnosis result, such as captured date, the capture position, and angle of a probe, and personal information of a patient. The personal information may include information that may affect a result of diagnosing breast cancer, such as, for example, the patient's age group, genetic information, previous history of breast cancer, weight, use of alcohol and/or tobacco, breast feeding history, family medical history, race, ethnicity, and gender.

In addition to capture information, the adaptive information of a diagnostic model may further include “performance information” that indicates performance history of a diagnostic model, such as diagnosis accuracy, supported by a statistical result. For example, a specific diagnostic model may include a statistical result that a benign tumor can be found through image data diagnosis at a relatively high possibility of 89%. Another diagnostic model may have a statistical result showing that a malignant tumor can be found through image data diagnosis at a decent-level possibility of 75%. Another diagnostic model may have a statistical result showing that a malignant tumor can be found through image data diagnosis at a relatively low possibility of 59%.

As described above, a diagnostic model used for diagnosis of image data is adapted using at least one additional information (e.g. capture information and diagnosis performance) added to the image data as adaptive information. Adapting a diagnostic model may be performed by selecting the most suitable diagnostic model among a plurality of diagnostic models based on adaptive information. Alternatively, adapting a diagnostic model may be performed by adjusting at least one parameter of a diagnostic model based on adaptive information.

A diagnostic model may include predetermined parameters. The diagnostic model may be any one of segmentation results, hierarchically classified results and learning results of an image set, which is equal to or greater than a specific size. The diagnostic model may include predetermined parameters that are optimized, for example, based on at least one of capture information and performance information. The diagnostic model may be adapted or learned during a diagnosis procedure, for example, based on at least one of capture information and performance information.

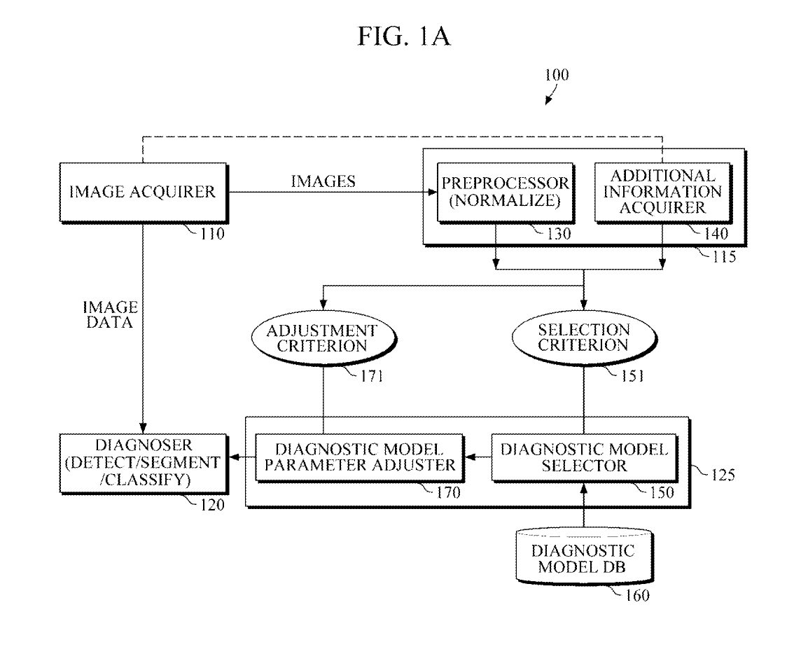

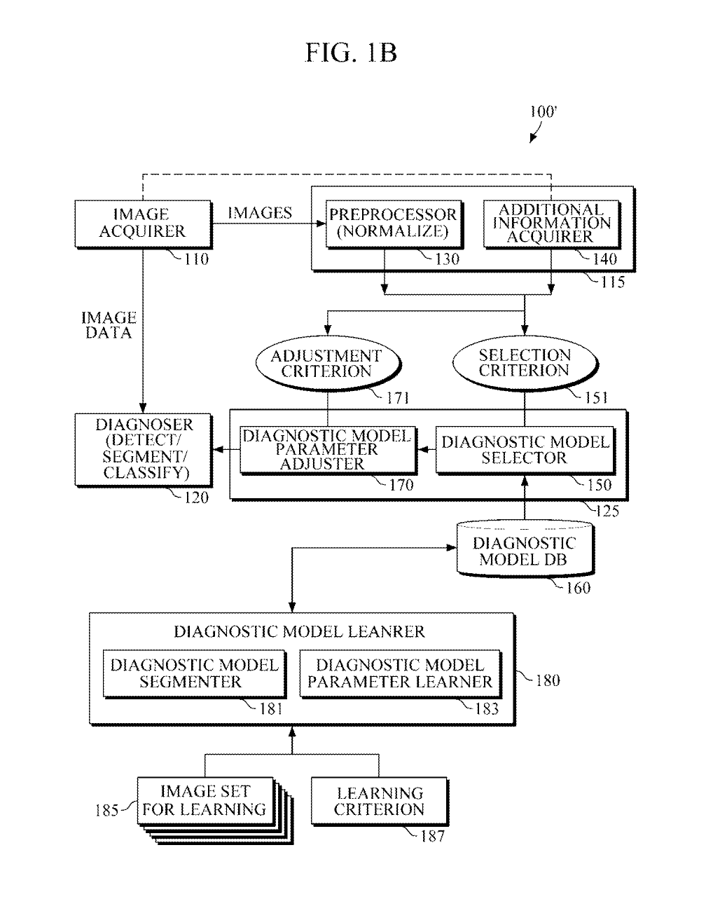

FIG. 1A is a diagram illustrating an example of a configuration of a system for adapting a diagnostic model for Computer-Aided Diagnosis (CAD).

Referring to FIG. 1A, a system 100 for adapting a diagnostic model for CAD includes an image acquirer 110, a diagnoser 120, an adaptive information provider 115, a diagnostic model adapter 125, and a diagnostic model database 160. While components related to the present example are illustrated in the system 100 for adapting a diagnostic model for CAD, it is understood that those skilled in the art may include other general components.

The image acquirer 110 may acquire images from an external image device. The images may be acquired, for example, from an ultrasound image scanner or a medical image system, such as, for example, a Picture Archiving and Communication System (PACS). The images may be ultrasound images that are acquired by scanning breasts of a specific patient for early diagnosis of breast cancer. Each ultrasonic image may not be an image of a full breast, but a specific part of the breast.

The image acquirer 110 may acquire various kinds of capture information in addition to the images. For example, capture information may include a medium used in acquiring images (e.g., ultrasound, X-ray, gamma ray), date of imaging, personal information of a patient to whom the object belongs to, and information on how the images are acquired. For example, information on how the images are acquired may include information, such as, for example, information on the area of the object that is captured, the angle at which the image was captured. The capture information may be provided to the adaptive information provider 115 as adaptive information. The capture information may be provided to the adaptive information provider 115 from a source different than the image acquirer 110. For example, capture information may be provided by a user's direct input of the capture information. In another example, capture information may be provided as a data file stored in an additional storage medium.

Apart from the capture information, performance information that indicates diagnosis performance on a diagnostic model may be provided to the adaptive information provider 115. In a non-exhaustive example, the performance information may be provided to the adaptive information provider 115 through an additional path.

The diagnoser 120 uses a common CAD technique to diagnose the images provided from the image acquirer 110. The diagnoser 120 may analyze the images using a diagnostic model to detect, segment and classify a Region of Interest (ROI).

The diagnostic model adapter 125 employs a diagnostic model used by the diagnoser 120. The diagnostic model adapter 125 may include a diagnostic model selector 150 and a diagnostic model parameter adjuster 170. The diagnostic model selector 150 may select the most suitable diagnostic model from among diagnostic models stored in the diagnostic model database 160 according to a selection criterion 151. The diagnostic parameter adjuster 170 may adjust a parameter of any diagnostic model, which is stored in the diagnostic model database 160 or selected by the diagnostic model selector 150, according to an adjustment criterion 171. The selection criterion 151 and the adjustment criterion 171 may be determined by adaptive information. The adaptive information may be provided by the adaptive information provider 115. The adaptive information provider 115 may provide capture information, such as preprocessed capture position data, and additionally acquired performance information to the diagnostic model adapter 125 as the selection criterion 151 and the adjustment criterion 171.

In the example shown in FIG. 1A, the adaptive information provider 115 may include a preprocessor 130 and an additional information acquirer 140. The preprocessor 130 may preprocess, for example, normalize capture information received from the image acquirer 110. The preprocessor 130 may provide the preprocessed capture information as adaptive information for a selection criterion 151 and an adjustment criterion 171. In addition, the additional information acquirer 140 may acquire additional information that shows a statistical result to indicate diagnosis performance of each diagnostic model stored in the diagnostic model database 160.

As shown in FIG. 1B, the diagnostic models stored in the diagnostic model database 160 may be learned by a diagnostic model leaner 180 using an image set 185 for learning and a learning criterion 187. The diagnostic model learner 180 may include a diagnostic model segmenter 181 configured to segment a diagnostic model, and a diagnostic model parameter learner 183 configured to learn a parameter of a diagnostic model according to the learning criterion 187.

Here, ‘learning’ refers to machine learning, and learning a diagnostic model composed of parameters is a process of optimizing the parameters of the diagnostic model using learning data.

FIG. 1B is a diagram illustrating an example of a configuration of the system 100 shown in FIG. 1A where a diagnostic model learner is included.

Referring to FIG. 1B, a system 100′ for adapting a diagnostic model for Computer-Aided Diagnosis (CAD) may include the image acquirer 110, a diagnoser 120, a diagnostic model adapter 125, an adaptive information provider 115, and a diagnostic model database 160, which are described above with reference to FIG. 1A. The above descriptions of the components described in FIG. 1A is also applicable to FIG. 1B, and is incorporated herein by reference. Thus, the above description may not be repeated here. The system 100′ for adapting a diagnostic model for Computer-Aided Diagnosis (CAD) may further include the diagnostic model learner 180.

The learning criterion 187 used by the diagnostic model learner 180 is information corresponding to adaptive information that is provided by the adaptive information provider 115. For example, the learning criterion 187 may include a probe's capture position and capture angle, a patient's personal information, and data that indicates the performance of a corresponding diagnostic model.

As described above, in order to perform CAD analysis of images, the system 100 may adapt a diagnostic model based on capture positions of the images, personal information of a patient, or information that indicates the performance of the diagnostic model. As a result, CAD may yields higher diagnosis accuracy.

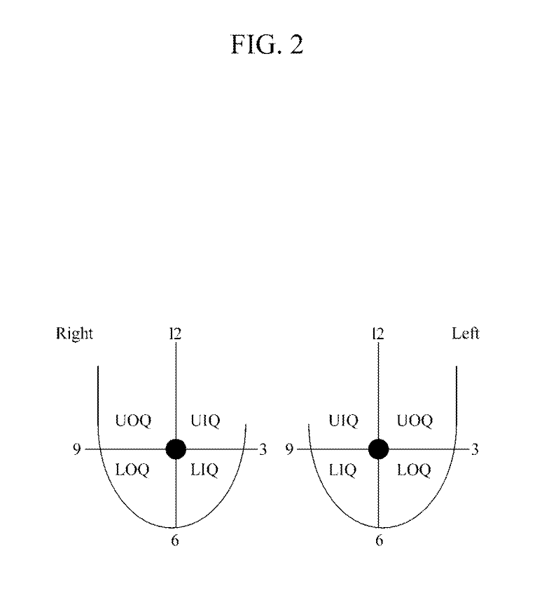

FIG. 2 is a diagram illustrating examples of classified capture positions included in capture information of images within a system for adapting a diagnostic model for CAD.

FIG. 2 is an example of the left and right breasts, each of which is divided into four areas around each nipple. In another example, the left and right breasts may be divided into three areas according to a level from a nipple. As such, variously standardized criterion may be applied to information indicating capture positions. A capture position for a specific image may be standardized to be a capture position for all images. The examples shown in FIG. 2 are merely illustrative, and it is apparent that other technique for classifying capture positions may be used.

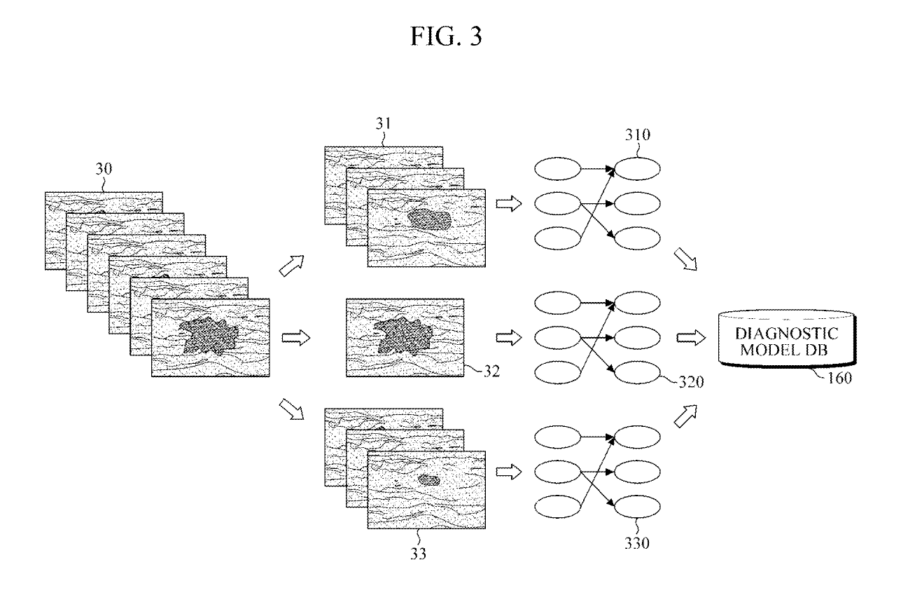

FIG. 3 is a diagram illustrating examples of a plurality of diagnostic models that are classified from a data set based on capture information within a system for adapting a diagnostic model for Computer-Aided Diagnosis (CAD).

Referring to FIG. 3, an image set 30 may be divided into three sub-image sets 31, 32, and 33 based on capture positions included in the capture information. For example, if images depict the captured images of breasts, the image set 31 may be a captured result of an UOQ area of the right breast shown in FIG. 2. The image set 32 may be a captured result of a nipple of the left breast shown in FIG. 2. The image set 33 may be captured results of a LOQ area of the left breast shown in FIG. 2.

As described above, an image set may be divided into sub-image sets 31, 32 and 33 according to capture positions and diagnostic models 310, 320 and 330 corresponding to the respective sub-image sets 31, 32, and 33 may be generated. The diagnostic models 310, 320, and 330 may be generated by a third party. Alternatively, the diagnostic models 310, 320, and 330 may be generated by learning an existing diagnostic model using the sub-image sets 31, 32, and 33. The diagnostic models 310, 320, and 330 generated as described above may be stored in the diagnostic model database 160.

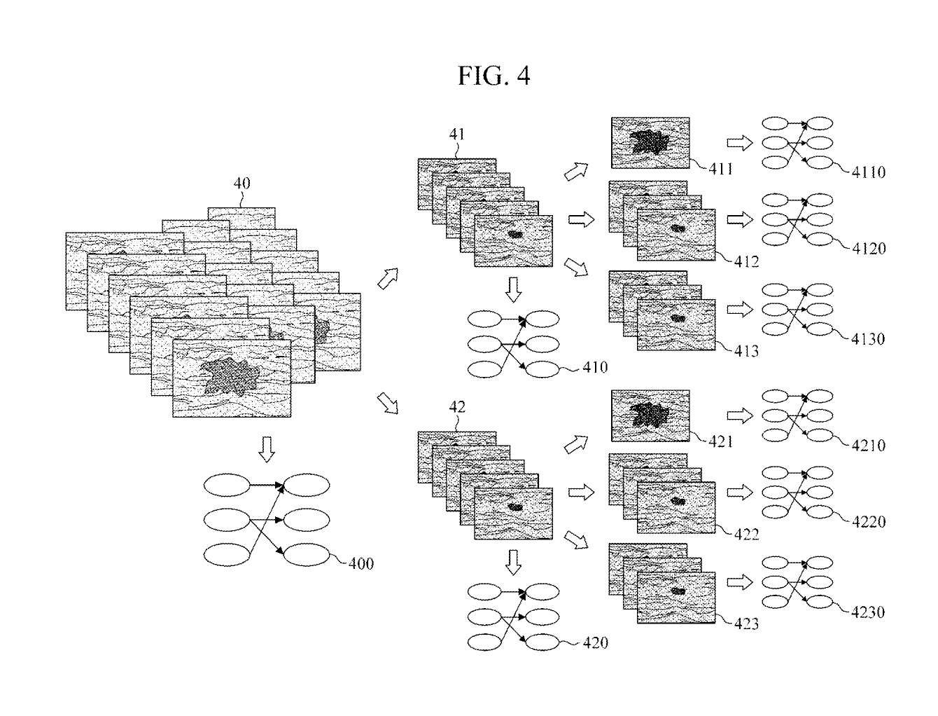

FIG. 4 is a diagram illustrating an example of a plurality of diagnostic models that are hierarchically classified with respect to an image set based on capture information within a system for adapting a diagnostic model for CAD.

Referring to FIG. 4, an image set 40 may be divided into two sub-image sets 41 and 42 based on capture positions included in the capture information. For example, if the imaging data is data of captured breasts, the sub-image set 41 may be an image set of a UOQ area of the right breast shown in FIG. 2, and the sub-mage data set 42 may be an image set of a nipple area of the left breast shown in FIG. 2.

Each of the sub-image sets 41 and 42 may be further divided into three sub-sub image sets according to patients' age groups contained in the capture information. In other words, the sub-image set 41 may be further divided into sub-sub data sets 411, 412 and 413 according to patients' age groups of the 20s, the 30s, and the 40s, respectively. The sub-image set 42 may be further divided into sub-sub image sets 421, 422, and 423 according to the patients' age groups of the 20s, the 30s, and the 40s, respectively.

After an image set is first divided based on capture positions and then further divided based on patients' age groups, diagnostic models corresponding to respective image sets before and after dividing may be generated. In other words, a diagnostic model 400 is generated for the image set 40. Diagnostic models 410 and 420 corresponding to the sub-image sets 41 and 42, respectively, are generated. Diagnostic models 4110, 4120, 4130, 4210, 4220, and 4230 corresponding to the sub-divided image sets 411, 412, 413, 421, 422, and 423, respectively, are generated.

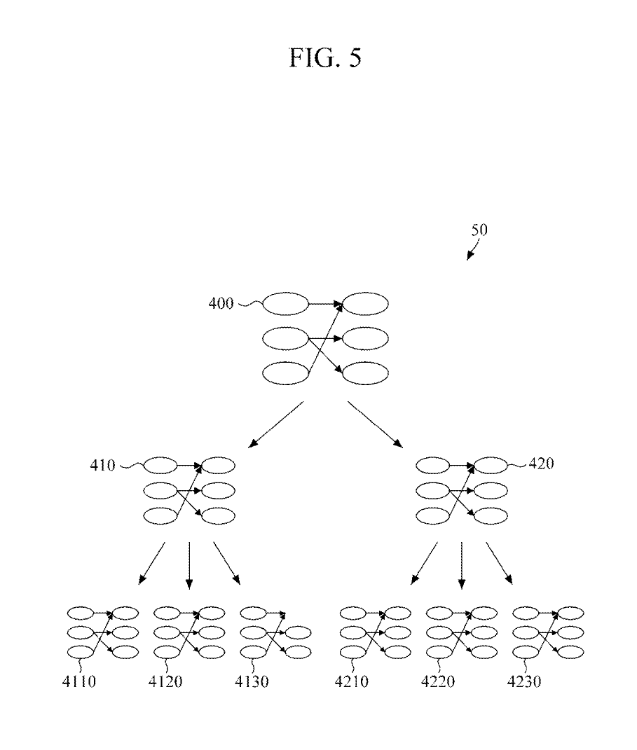

FIG. 5 is a diagram illustrating an example of a hierarchical structure of diagnostic models 50 stored in the system shown in FIG. 4.

Referring to FIG. 5, the diagnostic model 400, the sub-diagnostic models 410, and 420, and the sub-sub-diagnostic models 4110, 4120, 4130, 4210, 4220, and 4230 are connected hierarchically. Such diagnostic models are hierarchically structured because of the hierarchical structure of the corresponding image sets.

In the example shown in FIG. 5, the diagnostic model 400 is applied to every image data of the image set 40. However, the range of applicability of the sub-diagnostic models 410 and 420 is reduced to images that correspond to specific capture position information from among the image set 40. The range of applicability of the sub-diagnostic models 4110, 4120, 4130, 4210, 4220, and 4230 is further reduced to images that correspond to specific capture position information and to a specific age group.

In another example, a sub-diagnostic model and/or sub-sub diagnostic model may not be applied to an image set, if the image set is beyond a range of applicability thereof. Yet, if the image set is within a narrow range of applicability, a high-level of diagnosis accuracy can be expected.

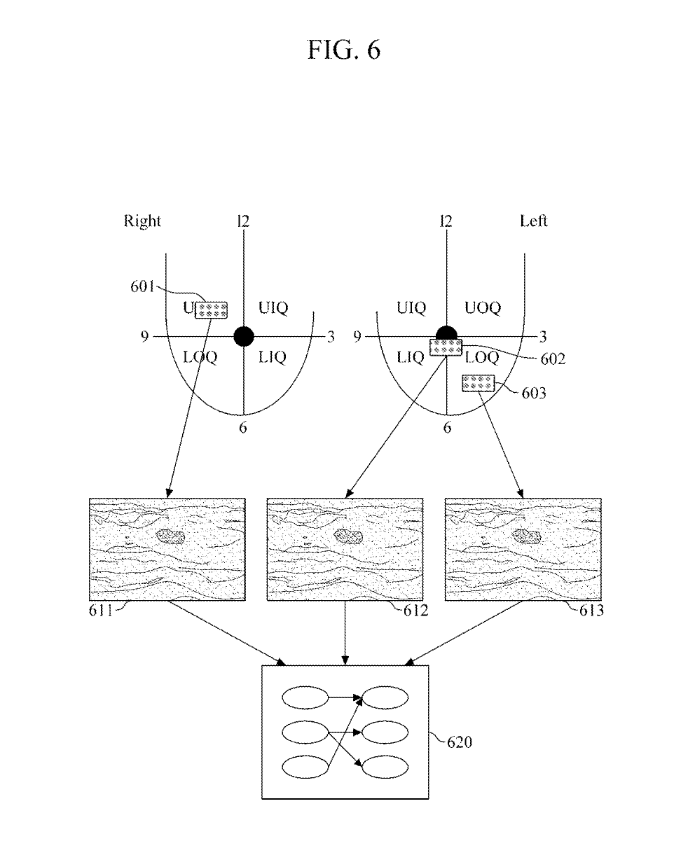

FIG. 6 is a diagram illustrating an example of performing diagnosis using a general diagnostic model for Computer-Aided Diagnosis (CAD).

Conventionally, one general diagnostic model for CAD is applied regardless of capture information. Referring to FIG. 6, in the prior arts, for example, the same diagnostic model 620 has been applied to an image set 611 captured at an UOQ area 601 of the right breast, image set 612 captured at a nipple area 602 of the left breast, and image set 613 captured at s LOQ area 603 of the left breast.

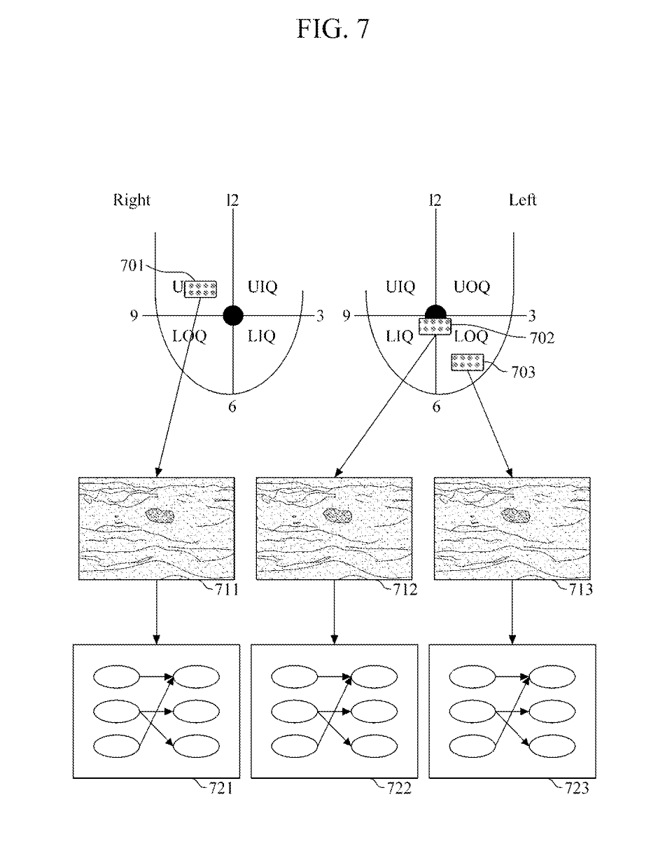

Unlike the prior art, different diagnostic models are applied according to capture positions in the examples described with reference to FIGS. 7 to 9.

FIG. 7 is a diagram illustrating an example of performing diagnosis using different diagnostic models according to capture positions in a system for adapting a diagnostic model for CAD.

According to an example, different diagnostic models may be selectively applied to different images, which are captured at different capture positions. For example, a diagnostic model 721 including parameters optimized for the UOQ area 701 may be selected to be applied for an image 711 captured at the UOQ area 701 in the right breast. As another example, a diagnostic model 722 including parameters optimized for the nipple area 702 may be selected to be applied for an image 712 captured at a nipple area 702 in the left breast. As another example, a diagnostic model 723 including parameters optimized for the LOQ area 703 may be selected to be applied for an image 713 captured at the LOQ area 703 in the left breast.

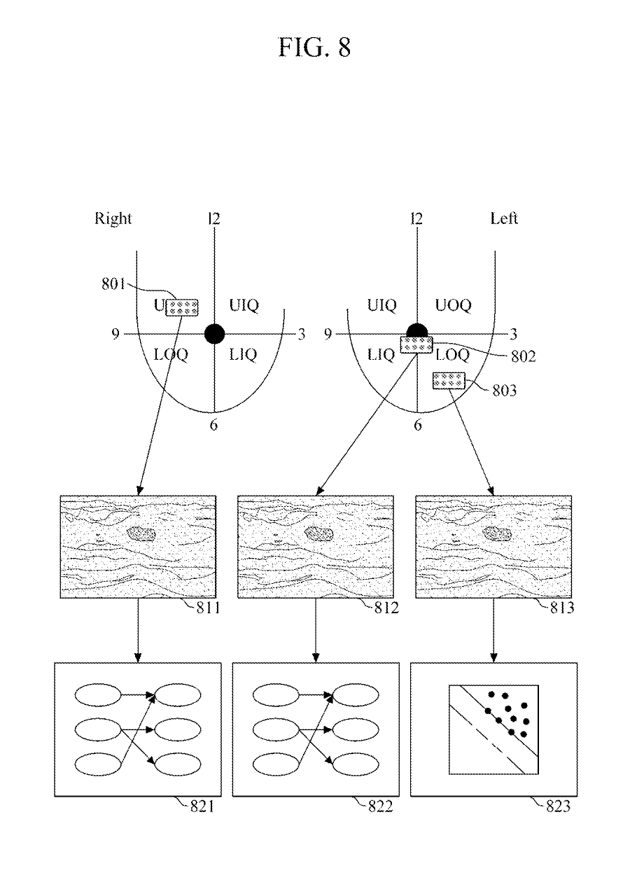

FIG. 8 is a diagram illustrating an example of performing diagnosis by adjusting parameters of a diagnostic model based on a capture position within a system for adapting a diagnostic model for CAD.

According to another example, a single diagnostic model may be selected for images captured at each of different capture positions. Then, parameters of the selected diagnostic model may be adjusted to optimize the selected diagnostic model for the capture position of each image. As a result, different diagnostic models may be applied to different images.

Referring to FIG. 8, for example, a diagnostic model is selected for an image 811 captured at an UOQ area 801 of the right breast. Parameters of the selected diagnostic model are adjusted for the UOQ area 801, so that the diagnostic model may be adapted into one optimized for the UOQ area 801. As another example, a diagnostic model is selected for an image 812 captured at a nipple area 802 of the left breast. Parameters of the selected diagnostic model are adjusted to be optimized for the nipple area 802, so that a diagnostic model 822 may be adapted into one optimized for the nipple area 802. As another example, a diagnostic model may be selected for an image 813 captured at a LOQ area of the left breast. Parameters for the selected diagnostic model are adjusted to be optimized for the LOQ area 803, so that a diagnostic model 823 may be adapted into one optimized for the LOQ area 803.

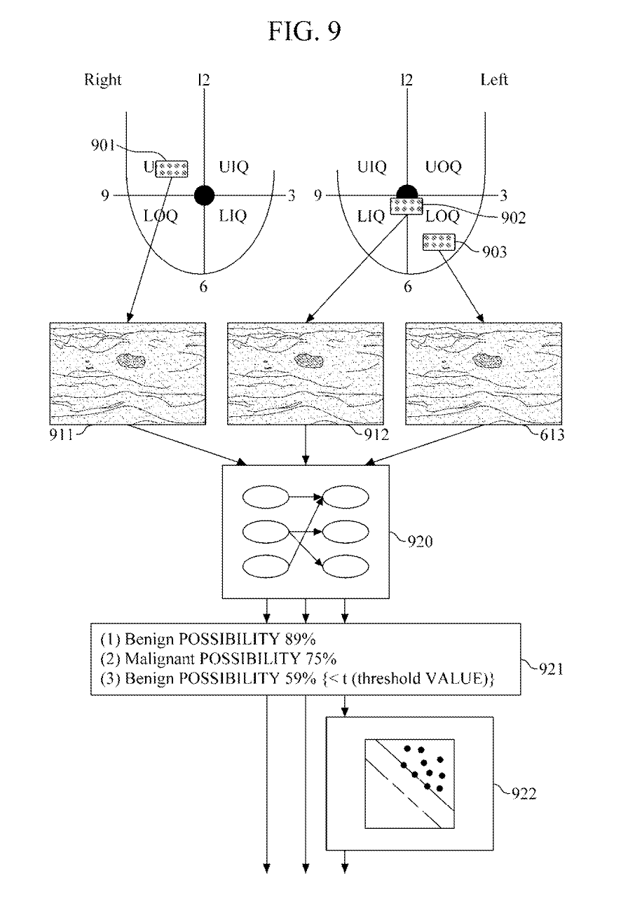

FIG. 9 is a diagram illustrating an example of performing diagnosis by determining whether to use a diagnostic model, based on a statistical result that shows diagnosis performance according to capture positions, within a system for adapting a diagnostic model for Computer-Aided Diagnosis (CAD).

According to the example shown in FIG. 9, a single diagnostic model is selected for images captured at different capture positions, and the diagnosis performance of the selected diagnostic model is determined. In response to a determination that performance of the diagnostic model is lower than a threshold value, a sub diagnostic model may be selected and applied. Referring to FIG. 9, for example, a single diagnostic model 920 may be selected, for example, for an image 911 captured at an UOQ area 901 of the right breast, an image 912 captured at a nipple area 902 of the left breast, and an image 913 captured at a LOQ area 903 of the left breast. Then, diagnosis performance of the diagnostic model 920 may be determined.

In FIG. 9, the diagnostic model 920 has diagnosis performance of 89% in detecting a benign tumor from the image 911 captured at the UOQ area 901 of the right breast. The diagnostic model 920 has diagnosis performance of 75% in detecting a malignant tumor from the image 912 captured at the nipple area 902 of the left breast. The diagnostic model 920 has diagnosis performance of 59% in detecting a malignant tumor from the image 913 captured at the LOQ area 903 of the left breast. It is determined whether the performance of each of the three diagnostic models exceeds a threshold value (e.g., a possibility of 60% to discover a benign/malignant tumor). As illustrated in the example of FIG. 9, when the performance of the diagnostic model 920 corresponding to the image 913 captured at the LOQ area 903 of the left breast is smaller than a threshold value, the diagnostic model may be further segmented. A sub-diagnostic model 922 with higher diagnosis performance may be selected.

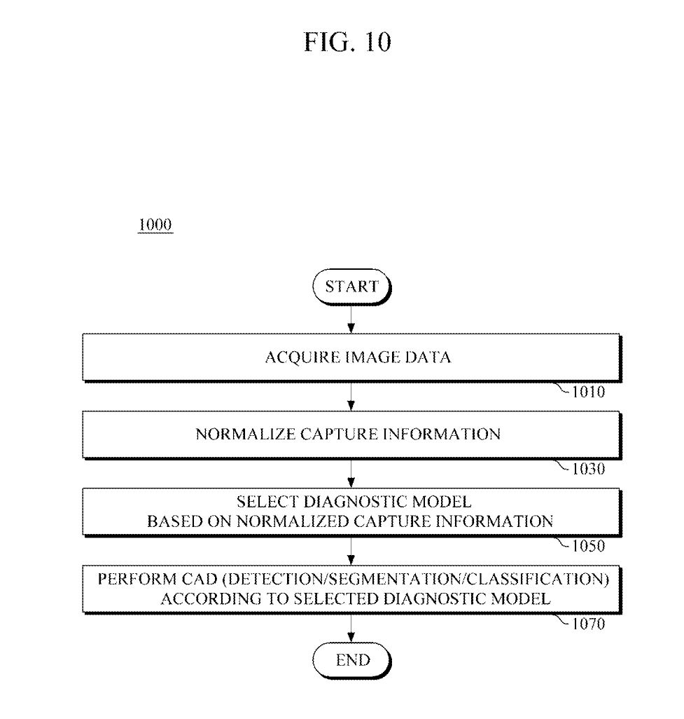

FIG. 10 is a diagram illustrating an example of a process of selecting a diagnostic model based on capture information according to an exemplary embodiment of a method for adapting a diagnostic model for CAD. The operations in FIG. 10 may be performed in the sequence and manner as shown, although the order of some operations may be changed or some of the operations omitted without departing from the spirit and scope of the illustrative examples described. Many of the operations shown in FIG. 10 may be performed in parallel or concurrently.

Referring to FIG. 10, in a method 1000 for adapting a diagnostic model for CAD, images are acquired in 1010 when a diagnosis process begins. In 1030, the capture information regarding the images, such as capture position information and probe angle information, is acquired and normalized. In 1050, using the normalized capture information, the most suitable diagnostic model may be selected among a plurality of diagnostic models. In 1070, CAD is performed on the images using the selected diagnostic model and then the CAD is terminated.

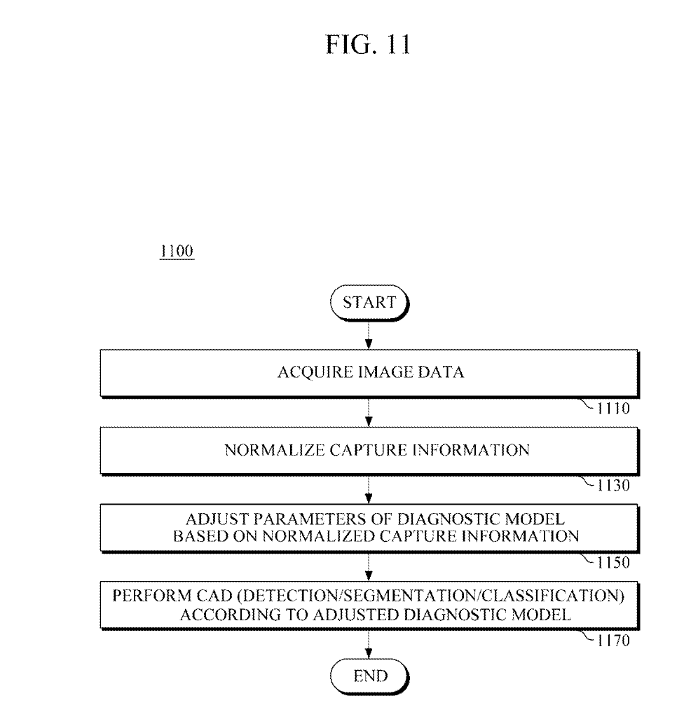

FIG. 11 is a diagram illustrating an example of a process of adjusting parameters of a diagnostic model based on capture information within a method for adapting a diagnostic model for CAD. The operations in FIG. 11 may be performed in the sequence and manner as shown, although the order of some operations may be changed or some of the operations omitted without departing from the spirit and scope of the illustrative examples described. Many of the operations shown in FIG. 11 may be performed in parallel or concurrently.

Referring to FIG. 11, in a method 1100 for adapting a diagnostic model for CAD, in 1110, images are acquired to commence a process of diagnosis. In 1130, capture information regarding the images, such as capture position information and probe angle information, is acquired and normalized. In 1150, using the normalized capture information, at least one parameter of a predetermined diagnostic model may be appropriately adjusted. In 1170, CAD is performed on the images using the diagnostic model with at least one adjusted parameter and the CAD is terminated.

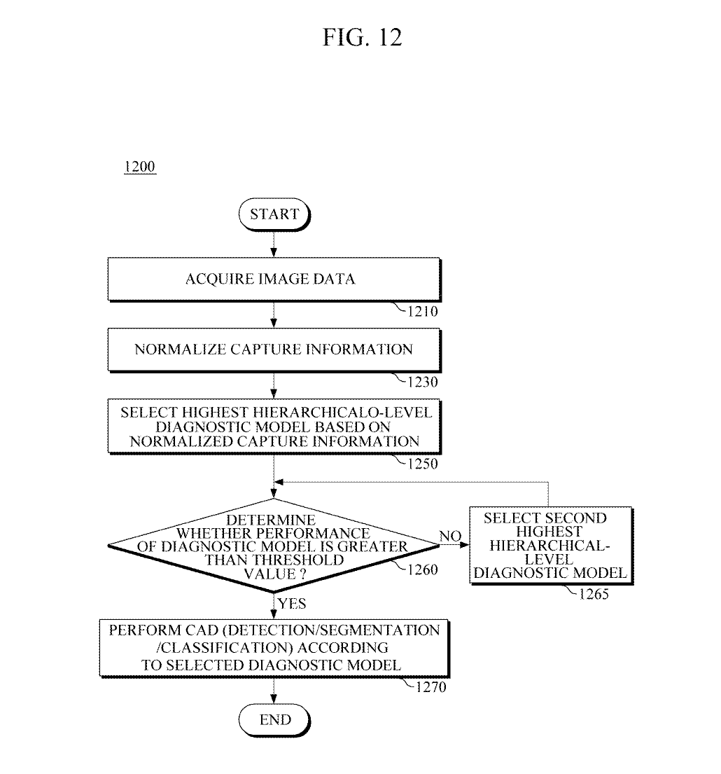

FIG. 12 is a diagram illustrating an example of a process of performing diagnosis by selecting a diagnostic model with decent diagnosis performance based on diagnosis performance of the diagnostic model within a method for adapting a diagnostic model for CAD. The operations in FIG. 12 may be performed in the sequence and manner as shown, although the order of some operations may be changed or some of the operations omitted without departing from the spirit and scope of the illustrative examples described. Many of the operations shown in FIG. 12 may be performed in parallel or concurrently.

Referring to FIG. 12, in a method 1200 for adapting a diagnostic model for CAD, in 1210, images are acquired to commence a process of diagnosis. In 1230, capture information regarding the images, such as capture position information and probe angle information, is acquired and then normalized. In 1250, using the normalized capture information, the highest hierarchical-level diagnostic model is selected from among a plurality of hierarchically classified diagnostic models. The highest hierarchical-level diagnostic model refers to a diagnostic model that can be applied to all images of a specific object, without regards to capture information such as, for example, a capture position. In 1260, it is determined whether performance of the selected diagnostic model is greater than a predetermined threshold value.

In response to a determination that the performance of the selected diagnostic model is greater than the predetermined threshold value, in 1270, CAD is performed on the images using the selected diagnostic model. In response to a determination that the performance of the selected diagnostic model is lower than the predetermined threshold value, in 1265, a diagnostic model with a level lower than that of the selected diagnostic model is re-selected. For the lower hierarchical-level diagnostic model, a process of checking diagnosis performance may be repeated in 1260. In this manner, a diagnostic model with acceptable diagnosis performance may be selected to be used for CAD.

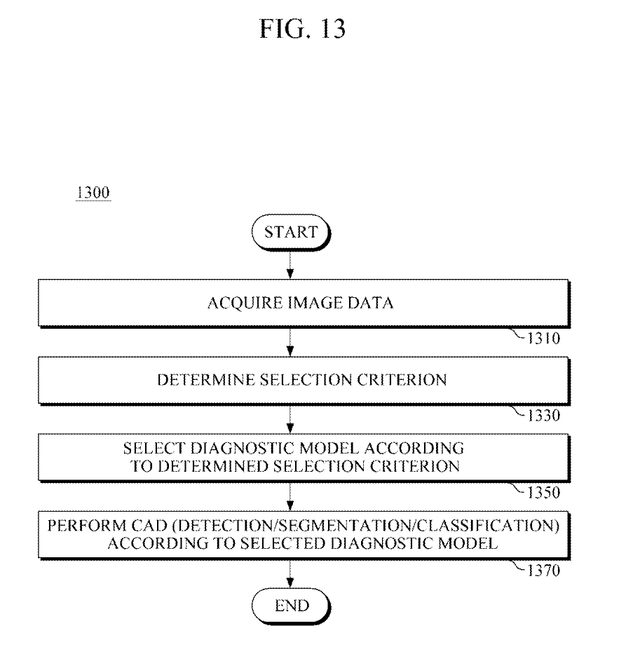

FIG. 13 is a diagram illustrating an example of a process of performing diagnosis by selecting a diagnostic model according to a determined selection criterion within a method for adapting a diagnostic model for CAD. The operations in FIG. 13 may be performed in the sequence and manner as shown, although the order of some operations may be changed or some of the operations omitted without departing from the spirit and scope of the illustrative examples described. Many of the operations shown in FIG. 13 may be performed in parallel or concurrently.

Referring to FIG. 13, in a method 1300 for adapting a diagnostic model for CAD, in 1310, images are acquired to commence a process of diagnosis. In 1330, a selection criterion is determined based on additional information regarding the images. The additional information may include information such as, for example, capture position information of the images, probe angle information of the images, personal information of a patient, and information that indicates performance of a diagnostic model. The selection criterion refers to a criterion used to select an optimized diagnostic model. In 1350, the most suitable diagnostic model may be selected among a plurality of diagnostic models using the selection criterion. In 1370, CAD is performed on the images using the selected diagnostic model and the diagnosis process may be terminated.

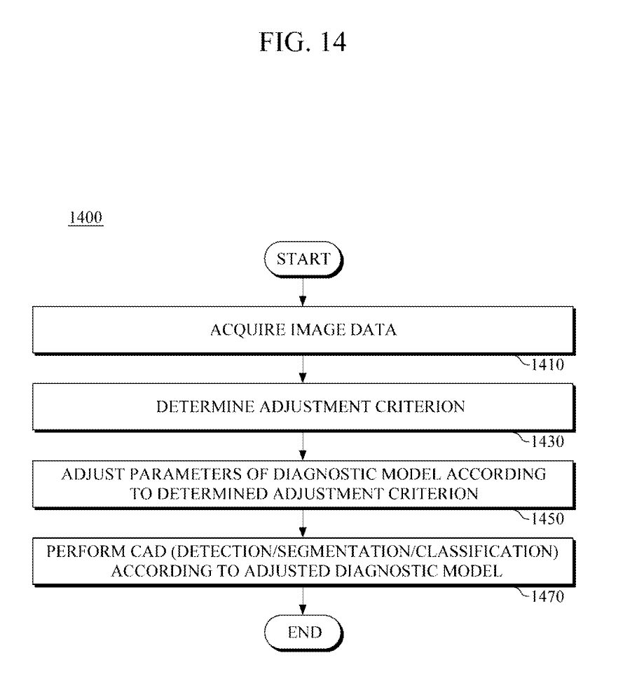

FIG. 14 is a diagram illustrating an example of a process of performing diagnosis by adjusting parameters of a diagnostic model according to a determined adjustment criterion within a method for adapting a diagnostic model for CAD. The operations in FIG. 14 may be performed in the sequence and manner as shown, although the order of some operations may be changed or some of the operations omitted without departing from the spirit and scope of the illustrative examples described. Many of the operations shown in FIG. 14 may be performed in parallel or concurrently.

Referring to FIG. 14, in a method 1400 for adjusting a diagnostic model for CAD, in 1410, images are acquired to commence a process of diagnosis. In 1430, an adjustment criterion is determined based on capture position information regarding the images, personal information of a patient, and performance information that indicates the performance of a diagnostic model. The adjustment criterion refers to a criterion used to optimally adjust parameters of a diagnostic model. In 1450, at least one parameter of a predetermined diagnostic model may be appropriately adjusted using the adjustment criterion. In 1470, CAD is performed on the images using the diagnostic model with at least one adjusted parameter, and then the diagnosis process may be terminated.

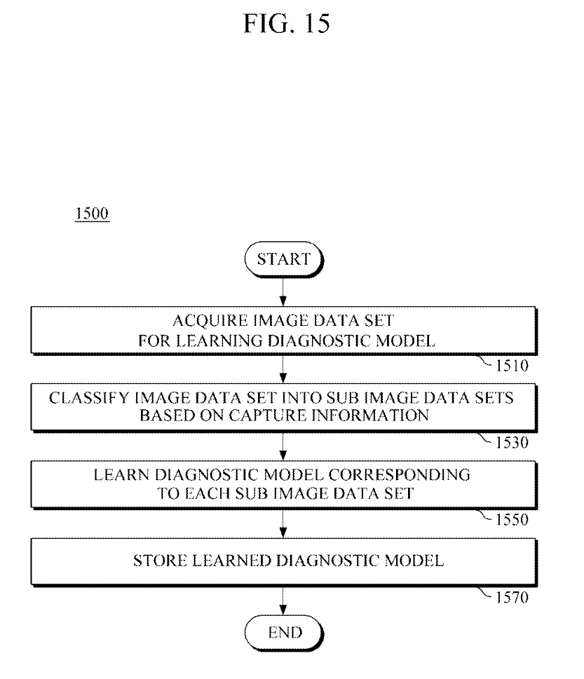

FIG. 15 is a diagram illustrating an example of a learning process of a diagnostic model within a method for adapting a diagnostic model for CAD. The operations in FIG. 15 may be performed in the sequence and manner as shown, although the order of some operations may be changed or some of the operations omitted without departing from the spirit and scope of the illustrative examples described. Many of the operations shown in FIG. 15 may be performed in parallel or concurrently.

Referring to FIG. 15, in a method 1500 for adjusting a diagnostic model for CAD, learning a diagnostic model begins in 1510, when an image set for learning a diagnostic model is acquired. In 1530, the image set is divided into a plurality of sub image sets based on capture information regarding the images, such as capture position information and probe information, and personal information of a patient. In 1550, the learning process may be performed by adjusting the diagnostic mode corresponding to each sub image set. In 1570, the learned diagnostic model is stored.

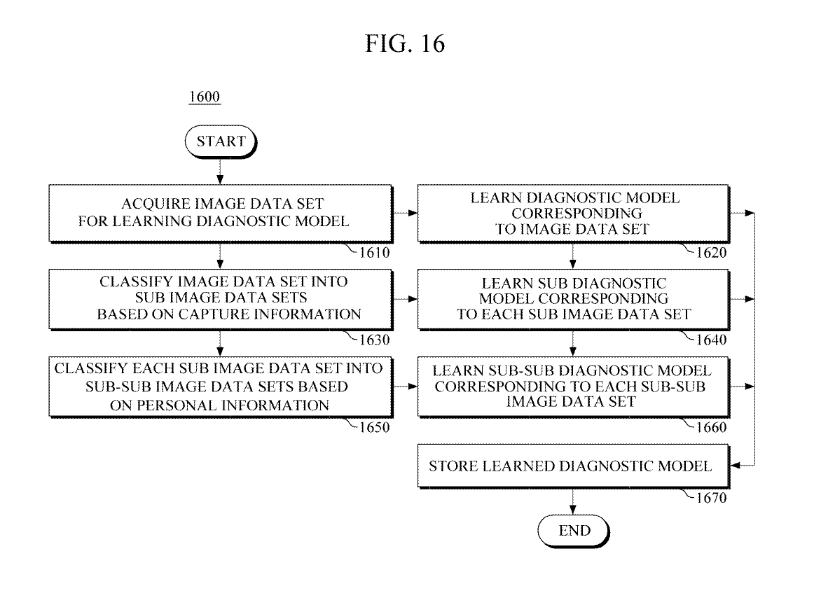

FIG. 16 is a diagram illustrating an example of a process of learning a hierarchically structured diagnostic model within a method for adapting a diagnostic model for CAD. The operations in FIG. 16 may be performed in the sequence and manner as shown, although the order of some operations may be changed or some of the operations omitted without departing from the spirit and scope of the illustrative examples described. Many of the operations shown in FIG. 16 may be performed in parallel or concurrently.

Referring to FIG. 16, in a method 1600 for adapting a diagnostic model for CAD, in 1610, learning a diagnostic model begins when an image set for learning a diagnostic model is acquired. In 1620, a diagnostic model corresponding to the image set is learned. In 1630, the image set is divided into a plurality of sub image sets based on capture information regarding the image, such as capture position information and probe information. In 1640, a diagnostic model corresponding to each sub image set may be adjusted to be learned. In 1650, each sub image set is further divided into a plurality of sub-sub image sets based on personal information regarding the image set, such as, for example, a patient's age group. In 1660, a sub-sub diagnostic model corresponding to each sub-sub image set is adjusted to be learned. The diagnostic models that have been learned through three hierarchical phases are stored. That is, in 1670, a diagnostic model corresponding to the image set, the sub-diagnostic models corresponding to respective sub-image sets, and the sub-sub diagnostic models corresponding to the respective sub-sub image sets are stored.

The processes, functions, and methods described above can be written as a computer program, a piece of code, an instruction, or some combination thereof, for independently or collectively instructing or configuring the processing device to operate as desired. Software and data may be embodied permanently or temporarily in any type of machine, component, physical or virtual equipment, computer storage medium or device that is capable of providing instructions or data to or being interpreted by the processing device. The software also may be distributed over network coupled computer systems so that the software is stored and executed in a distributed fashion. In particular, the software and data may be stored by one or more non-transitory computer readable recording mediums. The non-transitory computer readable recording medium may include any data storage device that can store data that can be thereafter read by a computer system or processing device. Examples of the non-transitory computer readable recording medium include read-only memory (ROM), random-access memory (RAM), Compact Disc Read-only Memory (CD-ROMs), magnetic tapes, USBs, floppy disks, hard disks, optical recording media (e.g., CD-ROMs, or DVDs), and PC interfaces (e.g., PCI, PCI-express, Wi-Fi, etc.). In addition, functional programs, codes, and code segments for accomplishing the example disclosed herein can be construed by programmers skilled in the art based on the flow diagrams and block diagrams of the figures and their corresponding descriptions as provided herein.

The apparatuses and units described herein may be implemented using hardware components. The hardware components may include, for example, controllers, sensors, processors, generators, drivers, and other equivalent electronic components. The hardware components may be implemented using one or more general-purpose or special purpose computers, such as, for example, a processor, a controller and an arithmetic logic unit, a digital signal processor, a microcomputer, a field programmable array, a programmable logic unit, a microprocessor or any other device capable of responding to and executing instructions in a defined manner. The hardware components may run an operating system (OS) and one or more software applications that run on the OS. The hardware components also may access, store, manipulate, process, and create data in response to execution of the software. For purpose of simplicity, the description of a processing device is used as singular; however, one skilled in the art will appreciated that a processing device may include multiple processing elements and multiple types of processing elements. For example, a hardware component may include multiple processors or a processor and a controller. In addition, different processing configurations are possible, such a parallel processors.

While this disclosure includes specific examples, it will be apparent to one of ordinary skill in the art that various changes in form and details may be made in these examples without departing from the spirit and scope of the claims and their equivalents. The examples described herein are to be considered in a descriptive sense only, and not for purposes of limitation. Descriptions of features or aspects in each example are to be considered as being applicable to similar features or aspects in other examples. Suitable results may be achieved if the described techniques are performed in a different order, and/or if components in a described system, architecture, device, or circuit are combined in a different manner and/or replaced or supplemented by other components or their equivalents. Therefore, the scope of the disclosure is defined not by the detailed description, but by the claims and their equivalents, and all variations within the scope of the claims and their equivalents are to be construed as being included in the disclosure.

What is claimed is:

1. A Computer-Aided Diagnosis (CAD) apparatus comprising:
an image acquirer configured to acquire an image of an object;
a diagnosis model store configured to store at least one diagnostic model;
an adaptive information provider configured to provide adaptive information regarding the image;
a diagnostic model adapter configured to select a diagnostic model from the diagnostic model store and to generate an adapted diagnostic model based on the selected diagnostic model and the adaptive information; and
a diagnoser configured to perform CAD on the image using the adapted diagnostic model.

2. The apparatus of claim 1, wherein the adaptive information comprises information indicating a position of the object.

3. The apparatus of claim 2, wherein the adaptive information further comprises personal information of the object that affects the CAD of the images.

4. The apparatus of claim 2, wherein the adaptive information further comprises information indicating the performance of a specific diagnostic model corresponding to the image captured at a specific capture position.

5. The apparatus of claim 1, wherein the diagnostic model adapter is further configured to adjust at least one parameter of the at least one diagnostic model based on the adaptive information and to provide the adjusted diagnostic model to the diagnoser.

6. The apparatus of claim 1, wherein
the diagnostic model store is further configured to store a plurality of diagnostic models that correspond to a plurality of image sets based on the adaptive information; and
the diagnostic model adapter is further configured to select one of the plurality of diagnostic models based on the adaptive information and to provide the selected diagnostic model to the diagnoser.

7. The apparatus of claim 6, wherein the diagnostic model adapter is further configured to adjust at least one parameter of the selected diagnostic model based on the adaptive information and to provide the adjusted diagnostic model to the diagnoser.

8. The apparatus of claim 6, wherein the plurality of diagnostic models are divided into two or more sub sets of diagnostic models based on the adaptive information, and the diagnostic models in each sub set are classified hierarchically based on the adaptive information, and
wherein the diagnostic models with greater amount of adaptive information are classified in a higher hierarchical level.

9. The apparatus of claim 8, wherein the diagnostic model adapter is further configured to:
select a highest hierarchical level diagnostic model among the plurality of diagnostic models based on a part of the adaptive information;
determine performance of the selected diagnostic model based on another part of the adaptive information;
in response to the performance of the selected diagnostic model not being suitable, select a lower hierarchical level diagnostic model and determine suitability of the lower hierarchical level diagnostic model; and
in response to the performance of the selected diagnostic model being suitable, provide the selected diagnostic model to the diagnoser.

10. The apparatus of claim 6, wherein the apparatus further comprises a diagnostic model leaner configured to:
classify images of an image set into sub image sets according to a learning criterion;
learn a diagnostic model stored in the diagnostic model store using the classified sub image sets; and
store the learned diagnostic model in the diagnostic model store.

11. A method for adapting a diagnostic model for Computer-Aided Diagnosis (CAD), the method comprising:
acquiring an image of an object;
providing adaptive information regarding the images;
acquiring a diagnostic model for diagnosis of the image;
adapting, at a diagnostic model adapter, the diagnostic model based on the adaptive information; and
performing CAD on the image using the adapted diagnostic model.

12. The method of claim 11, wherein the adaptive information comprises information indicating a position of the object.

13. The method of claim 12, wherein the adaptive information further comprises personal information of the object that affects the CAD of the images.

14. The method of claim 12, wherein the adaptive information further comprises information indicating the performance of a specific diagnostic model for the image captured at a specific capture position.

15. The method of claim 11, wherein the adapting of the diagnostic model comprises adjusting at least one parameter of the diagnostic model based on the adaptive information and providing the adjusted diagnostic model.

16. The method of claim 11, wherein the adapting of the diagnostic model comprises selecting one of a plurality of diagnostic models that correspond to a plurality of image sets based on the adaptive information and providing the selected diagnostic model.

17. The method of claim 16, wherein the adapting of the diagnostic model comprises adjusting at least one parameter of the selected diagnostic model and providing the adjusted diagnostic model.

18. The method of claim 16,
wherein the plurality of diagnostic models are classified into two or more sub sets of diagnostics models based on the adaptive information, and the diagnostic models in each sub set are hierarchically classified according to the adaptive information, and
wherein the diagnostic models with greater amount of the adaptive information are classified in a higher the hierarchical level.

19. The method of claim 18, wherein the adapting of the diagnostic model comprises:
selecting a highest hierarchical level diagnostic model from among the plurality of diagnostic models based on a part of the adaptive information;
determining performance of the selected diagnostic model based on a another part of the adaptive information;
in response to the performance of the selected diagnostic model not being suitable, selecting another diagnostic model with a lower hierarchical level than the hierarchical level of the selected diagnostic model and determining the suitability of the lower hierarchical level diagnostic mode; and
in response to the performance of the selected diagnostic model being suitable, providing the selected diagnostic model.

20. The method of claim 16, further comprising:
classifying an image set according to a learning criterion;
learning a diagnostic model stored in the diagnostic model store using the classified image set; and
storing the learned diagnostic model to use the learned diagnostic model in the adapting of the diagnostic model.

21. A diagnostic model provider for a Computer-Aided Diagnosis (CAD) system comprising:
an adaptive information provider configured to provide adaptive information regarding images of an object;
a diagnostic model store configured to store a plurality of diagnostic models corresponding to the images; and
a diagnostic model adapter configured to select one of the plurality of diagnostic models based on the adaptive information and to provide the selected diagnostic model for the CAD,
wherein the plurality of diagnostic models are classified hierarchically based on the adaptive information and the diagnostic models with greater number of adaptive information are classified in a higher hierarchical level.

22. The diagnostic model provider of claim 21, wherein the diagnostic model adapter is further configured to:
select a highest hierarchical level diagnostic model among the plurality of diagnostic models based on a part of the adaptive information;
determine performance of the selected diagnostic model based on another part of the adaptive information;
select a lower hierarchical level diagnostic model, in response to the performance of the selected diagnostic model being lower than a threshold value; and
provide the selected diagnostic model for the CAD, in response to the performance of the selected diagnostic model being higher than a threshold value.
